(19) United States
(12) Patent Application Publication
(10) Pub. No.: US 2011/0082545 A1
(43) Pub. Date: Apr. 7, 2011

(54) DRUG ELUTING BREAST IMPLANT COVER OR COATING

(75) Inventor: Robert M. Freund, Old Westbury, NY (US)

(73) Assignee: LIPOSE CORPORATION, Greenwich, CT (US)

(21) Appl. No.: 12/773,281

(22) Filed: May 4, 2010

Related U.S. Application Data
(60) Provisional application No. 61/247,595, filed on Oct. 1, 2009.

(51) Int. Cl. A61F 2/12

(52) U.S. Cl. 623/8

(57) ABSTRACT

A biodegradable cover or coating for a breast implant includes a biodegradable cover or coating over the breast implant and contains one or more drugs for delivery at the surgical site, particularly for treating or preventing infection, pain, inflammation, capsular contracture, scarring or other indication or complication associated with breast augmentation or breast reconstruction.

### CROSS-REFERENCE TO RELATED APPLICATION

This application claims the benefit under 35 U.S.C. §119(e) to U.S. Provisional Patent Application No. 61/247,595, filed Oct. 1, 2009, the entire disclosure of which, in its entirety, is hereby incorporated by reference herein.

### BACKGROUND OF THE INVENTION

The present invention relates to a biodegradable cover or coating for a breast implant includes a biodegradable cover or coating over the breast implant and contains one or more drugs for delivery at the surgical site, particularly for treating or preventing infection, inflammation, capsular contracture, scarring or other indication or complication associated with breast augmentation or breast reconstruction.

Breast enhancement is commonplace in society. More than 600,000 breast augmentation procedures are performed in the U.S. each year. Common problems involving breast implants include implant infection and capsular contracture, among other things.

Another issue arising with the use of breast implants is the formation of excess scar tissue around an implant. Such tissue can harden and lead to tightening around or squeezing of the implant, a phenomenon known as capsular contracture. While scar tissue and capsule formation is a normal process, when scar tissue is excessive the subsequent capsule is called capsular contracture and can lead to an implant that is misshapen, painful, hard and can attain an unnatural appearance and feel. Additionally, capsular contracture appears to be more common following infection, hematoma and seroma.

The reasons for implant infection in the early post-operative period can include residual blood following surgery, direct contact with breast ducts (that have bacteria within their lumens) that have been injured during surgery or other surgical contaminants. Current treatment includes antibiotic washings of the surgical site and implants at the time of surgery and oral antibiotics after surgery.

Infection can occur anytime from several days to several years after implantation; however, it occurs more frequently in the immediate post-operative period. Acute infection is diagnosed when the patient exhibits pain, fever, and tenderness around the implant usually manifested within several days from the time of surgery.

The reason for capsular contracture is not well-defined. However, several different treatments have yielded improved results with less chance of capsular formation. First, leukotriene receptor antagonists, such as zafirlukast (Accolate®), montelukast (Singulair®), and pranlukast administered orally have demonstrated the ability to moderate the capsular contracture. Second, antibiotics surrounding the implant have anecdotally been a source of improvement in capsular contracture. Finally, textured implants have yielded a reduction in capsule formation, but is not desirable because of aesthetic issues.

Implant infection is most commonly attributed to contamination of the sterile field during surgery or to contamination arising from lymph node or mammary duct dissection during surgery. Bacteria can migrate deep within the breast tissue via the mammary ducts. Incision through the ducts during subglandular placement thus opens a temporary but direct external route for contamination of the implant after placement. Bacteria colonized from the mammary ducts and nipples is similar to exogenous flora found on the skin, namely coagulase negative Staphylococcus, P. acne, and Bacillus subtillus.

Subclinical infection is perceived to be a contributor to capsular contracture. Subclinical infection is defined as bacterial colonization of a surface with or without biofilm formation. It does not produce the signs and symptoms traditionally associated with frank infection (such as pain, tenderness, fever, and pus) and manifests itself as a chronic inflammatory response. This inflammatory response leads to an overaggressive collagen deposition during tissue remodeling that leads to fibrous tissue buildup and capsule rigidity with eventual implant distortion.

Most surgeons engage in prophylactic efforts to reduce the incidence of infection associated with breast implants, including a no-touch technique by the surgeon. For example, in addition to meticulous attention to sterility, many surgeons irrigate the implant pocket and bathe the implant itself with gentamycin, cefazolin, povidone-iodine or another antibiotic solution. Post-operative counseling includes instructing the patient to neither touch the incision sites nor to immerse them in hot water for at least two weeks (or until healing is complete). Prophylactic oral antibiotics can also be given to patients prior to, during and after surgery to prevent post-implant colonization. Additionally, implant placement below the muscle avoids (or at least minimizes) surgical contact with the mammary ducts.

Adams and colleagues used a method for reducing capsular contracture caused by bacterial implant colonization. They provided an antibacterial irrigation solution and employed sterile technique prior to and during surgery. Adams' “triple antibiotic solution” originally contained a mixture of bacitracin, gentamycin, and cefazolin and was shown to be active against bacteria most commonly known to colonize breast implants. Adams subsequently published results of a six-year clinical study showing that patients who received surgeries incorporating these techniques have a 1% capsular contracture rate as opposed to national rates, which approached 15-20% in that same time period (Adams et al. (2006) Plast. Reconstr. Surg. 117:30-36).

Texturing the outside silicone surface has been employed as a technique to prevent capsular contracture. The textured surface causes a disorganization of collagen during deposition which minimizes capsule contracture. However, these implants have not significantly penetrated the market because, in use, tissue adherence to the textured surface leads to visible dimpling effect when the recipient moves. Textured implants also tend to have thicker shells than smooth implants and higher rupture rates.

The partial or total adhesion of the implant to the capsule due to such tissue ingrowth may be undesirable in the event it becomes necessary to remove or replace the implant. Notwithstanding the foregoing disadvantages, textured implants having a biocompatible, non-bioabsorbable outer tissue-contacting surface are generally considered to reduce the incidence of capsular contracture in patients.

There remains a need for an implantable filled breast prosthesis, typically filled with silicone gel or saline solution, with or without gas pockets, that resists capsular contracture following implantation and that resists adherence of the implant to the capsule.

The biodegradable covers or coatings of the present invention when used with breast implants overcome these drawbacks while reducing or preventing capsular contracture as well as treating or preventing infection, pain, inflammation, scarring or other indications or complications associated with breast augmentation or breast reconstruction. As used herein, the generic term “covering” means a cover formed separately from a breast implant into which a breast implant may be inserted or a coating formed upon the surface of the breast implant. At times the terms “cover” and “coating” are used herein along with the generic term “covering,” and unless a specific distinction is made between a cover and a coating, the details of the invention relating to coverings generally relate both to covers and coatings.

Also, as used herein, the singular forms “a”, “an”, and “the” include plural referents, and plural forms include the singular referent unless the context clearly dictates otherwise.

### BRIEF SUMMARY OF THE INVENTION

One aspect of the present invention relates to a biodegradable covering for a breast implant, the covering comprising at least one biodegradable polymer layer dimensioned and shaped to cover the breast implant, the covering containing at least one drug to at least minimize at least one of infection and capsular contraction.

The present invention is directed to a biodegradable covering for breast implants. In certain embodiments, the covering comprises one or more biodegradable polymer layers dimensioned and shaped to cover the breast implant. Such covering has an outer surface that may be smooth or textured, as desired, to engage tissue and an inner surface directed toward the breast implant. The implant can be inserted into the covering or the covering can be formed around the implant. Preferably, where the covering is in the form of a separate cover, it is sealed, preferably after the implant is bathed in an antibiotic solution and inserted into the cover. In other embodiments, the cover may be in the form of a sheet, film, or mat of one or more biodegradable polymer layers that can be wrapped or cut into the dimension and shape of a breast implant and thereby used to cover at least a portion of the breast implant, for example, in an annular fashion around the periphery of the breast implant shell.

In other embodiments, the implant may be coated with an appropriate drug-eluting coating to be described hereinafter that chemically or physically bonds to the implant and is bioabsorbable or biodegradable in the body, such that the coating dissolves over time.

The covering of the invention can inhibit or reduce formation of scar tissue in and around the implant and/or inhibit or reduce capsular contracture in and around the implant in a patient. When drugs are present in the polymer matrix that forms the covering, such drugs can elute into the surrounding tissue to inhibit, prevent or treat any bacterial infection or colonization for antimicrobial agents. Preferably, the drug is at least one antibiotic. Other drugs will also beneficially affect scarring and capsular contracture formation by interfering with collagen formation. Such other drugs include leukotriene receptor antagonists or inhibitors and/or calcium channel blockers. Other drugs may be used to treat other anticipated conditions, based on the patient and the patient's circumstances.

For example, the covering containing appropriate antibiotics can provide protection against colonization by bacteria until a complete capsule can form to block the mammary ducts that blocks bacterial colonization. The covering completely surrounding the implant provides a sterile barrier around the implant that can both kill any contaminating bacteria from the surgical insertion itself as well as elute antimicrobial agents and/or other drugs after surgery to prevent bacterial migration to the implant during the healing process and to reduce and preferably eliminate capsular contracture.

The covering of the invention can comprise multiple layers, typically from one to five layers. In one embodiment, the covering can include an inner layer facing the breast implant shell optionally containing at least one drug, one or more central polymeric layers that can optionally contain at least one drug, and an outer polymeric layer that can have a smooth or textured outer surface and optionally contain at least one drug, so long as at least one of the layers, and preferably all of them contain at least one drug. The different layers can contain the same or different drugs which can elute at different times based on the dissolution rate of the layers containing them. The polymeric inner layer, and any intermediate and outer layers of the covering can be made of the same or different biodegradable polymers.

Any of the covers or coatings of the invention should completely encase the breast implant.

The polymeric cover or coating is made from a biodegradable polymer.

Another aspect of the invention is a breast implant assembly comprising a biodegradable covering of the invention containing a breast implant.

Yet another aspect of the invention relates to a method for reducing post-surgical indications or complications, such as capsular contracture or infection, from breast augmentation or breast reconstruction in a subject, the method comprising surgically implanting into the subject a breast implant assembly of the invention.

### DETAILED DESCRIPTION OF THE PREFERRED EMBODIMENTS

As noted above, the present invention is directed to a biodegradable elastomeric covering for breast implants. The covering in the form of a cover has an appropriate form-fitting shape for the implant and may encase an implant in the operating room prior to its insertion during surgery. The covering in the form of a cover can also be preassembled with the breast implant and supplied for surgery as a breast implant assembly, where the term “breast implant assembly” also includes a covering supplied for surgery in the form of a coated breast implant.

In some embodiments, the biodegradable covering has substantially the same shape and size as the implant itself (e.g., round, teardrop, contoured, anatomical and the like), and completely covers the implant. In some embodiments, the covering is shaped like a shower cap and may be sealed after the implant is inserted into the covering and the covering shrunk around the implant to provide a closely fitting covering. The biodegradable covering of the invention is useful to reduce capsular contracture and to deliver drugs into the surrounding tissue, to facilitate healing or to reduce and preferably prevent infection, pain, and/or other conditions, indications or complications associated with breast implants.

The covering acts as a carrier of the pharmaceutical agents, and may be a repeating structure of amino acids, such as tyrosine, leucine or arginine, or other biodegradable polymeric materials. In the coating embodiment, the coating chemically bonds to the surface of the implant, typically made of silicone, or physically is retained within the interstitial pores of the surface of the implant. The amino acid or other structure will be designed to adhere to the surface of the silicone implant and carries at least one drug or pharmaceutical agent. It will dissolve or be degraded over a well-defined period of time.

The covering preferably has a substantially uniform thickness over the breast implant wall, and may be one or more biodegradable polymer layers. In the cover embodiment, the implant is inserted into the covering through a suitable aperture, which preferably may be a slit that can readily be overlapped to fully cover the implant so as to minimize and preferably prevent infection to an uncovered portion of the implant. Those of skill in the art can readily determine appropriate shapes, sizes and configurations for the apertures or slits for a given size and shape of breast implant.

Methods of preparing the polymer layers for the covering of the invention include dip molding, spray coating, single stream or dual stream electrospinning which use nanofibers, and other methods which are conventional or known in the art. Materials for the molds for covers are conventional, such as plastics, like polypropylene, nylon, polytetrafluoroethylene, delrin; metals like stainless steel and titanium; or glass or ceramics. Those skilled in the art could readily obtain or fabricate molds or other equipment compatible with the polymer used to make the cover, which of course must be readily removable from the molds.

Any of the covering embodiments of the invention can be a single layer or multiple layers of any suitable thickness, and preferably a substantially uniform thickness, except where a textured layer or layers are desired. The covering containing the drug or drugs to be eluted should be sufficiently thick to control the time of elution before the covering layer or layers are dissolved within the subject receiving the implant with the covering of the invention. In multilayered embodiments, the layers, some of which may contain and therefore elute different drugs, typically are formed or applied serially one on the other. The thickness of a layer typically depends on viscosity, which in turn depends on polymer concentration in a solution used to make the covering. Thickness of the covering or coating can be increased by increasing the number of dips, sprays or other techniques used to make the covering.

Certain physicomechanical properties of the covering of the invention are similar to those of the implant shell. For example, at body temperature, both the shell and the covering are soft and malleable. The covering in the form of a cover is also elastomeric so that it can be stretched around the implant, or can be stretched and will shrink to fit snugly around the implant. Alternatively such a covering can be made entirely of a fibrous, knit, woven, or non-woven construction to impart softness and flexibility to readily house the breast implant.

The covering of the invention is formed from one or more biodegradable polymeric layers, at least one of which contains at least one drug. The covering, particularly in the form of a coating, may be formed from any number of clathrate compounds known to those skilled in the art. Clathrate compounds are chemical substances that may hold other chemical compounds, such as the drugs or biologically active agents used in the present invention. Clathrates are also known as host-guest complexes, inclusion compounds, and adducts. Methods of making clathrates and biodegradable polymers are well known in the art and are adaptable to the use of the covering of this invention by no more than routine experimentation.

As used herein, a “biodegradable polymer” is a biocompatible polymer that is hydrolytically labile, oxidatively labile, or susceptible to enzymatic action, or any combination thereof, which action leads to the degradation or absorption of the polymer within the body of the subject in which it is implanted. Biodegradable polymers have various times for degradation and dissolution, which can depend, for example, on the nature and size of the breakdown products.

The biodegradable polymer used in the present invention also is biocompatible. A biocompatible polymer is a polymer which is compatible with living tissue or a living system and is acceptable for use in or by animals or humans. Thus, a biocompatible polymer does not cause physiological harm to any significant or unacceptable degree, does not cause any or any significant amount of inflammation or immunological reaction, and is not toxic or injurious to the living tissue or system. For example, a biocompatible polymer can be ingested, implanted, placed on or otherwise used in a living subject or tissue without adverse effects.

Many biodegradable polymers are suitable for use in producing the covering of the invention. In selecting polymers for use in the invention, the glass transition temperature (Tg) of the polymers, as well as the polymer-drug combination can be considered along with other parameters. For example, polymers with sufficiently low Tg can be pressed into films at low temperatures. Since some drugs may decompose at high temperature, a low Tg polymer offers the ability to use thermal methods even in the presence of drugs. As used herein, low Tg polymers are those having a Tg below 40° C. The covering of the invention in the form of films, e.g., as are prepared by dip coating, desirably have a Tg in of about 20° C. to about 30° C., but can vary from as low as about 10° C. up to about body temperature or even to about 40° C. These Tg values are for the final formulation of the covering (including polymer, drug or any other ingredient), as it is well known that adding excipients (e.g., drugs or plasticizers) to polymers can either lower or increase the Tg.

One way to assess whether a covering in the form of a cover has sufficient flexibility for use in the invention is to measure the elongation of the polymer. Suitable films have an elongation at yield of about 10% to about 400%, such that films are generally too stiff if elongation is below 10% and too pliable if above 400%.

Polymers with high glass transition temperatures tend to be stiff and if made into films, would be too stiff for use in the covering for the breast implants in such a form. In such cases, incorporation of drugs can lower glass transition temperatures, making the stiffer polymers softer and more suitable for use. Alternatively, these high Tg polymers remain useful for the invention since they can be nanospun into felts for formation into a covering of the invention. Such felts render the covering soft even if the polymer itself may be stiff when formed into a film.

It is within the skill of the art to select polymers, drugs and processing methods to prepare the covering and incorporate at least one drug into the covering of the invention.

Exemplary biodegradable polymers suitable for use in the invention include but are not limited to:

polylactic acid, polyglycolic acid and copolymers and mixtures thereof, such as poly(L-lactide) (PLLA), poly(D,L-lactide) (PLA), polyglycolic acid or polyglycolide (PGA), poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), poly(D,L-lactide-co-caprolactone) (PLA/PCL) and poly(glycolide-co-caprolactone) (PGA/PCL);

poly(oxa)esters, polyethylene oxide (PEO), polydioxanone (PDS), polypropylene fumarate, poly(ethyl glutamate-co-glutamic acid), poly(tert-butyloxy-carbonylmethyl glutamate), polycaprolactone (PCL), polycaprolactone co-butylacrylate, polyhydroxybutyrate (PHBT) and copolymers of polyhydroxybutyrate, poly(phosphazene), poly(phosphate ester), poly(amino acid), polydepsipeptides, maleic anhydride copolymers, polyiminocarbonates, poly[(97.5% dimethyl-trimethylene carbonate)-co-(2.5% trimethylene carbonate)], poly(orthoesters), tyrosine-derived polyarylates, tyrosine-derived polycarbonates, tyrosine-derived polyiminocarbonates, tyrosine-derived polyphosphonates, polyethylene oxide, polyethylene glycol (PEG), polyalkylene oxides (PAO), hydroxypropylmethylcellulose, polysaccharides such as hyaluronic acid, chitosan and regenerate cellulose, and proteins such as gelatin and collagen, and mixtures and copolymers thereof, and

polyester urethane urea (PEUU).

Other suitable biodegradable polymers and copolymers may also be used, as well as polyethylene glycol (PEG) derivatives or blends of any of the foregoing. All such polymers which provide the desired flexibility, pliability and/or softness to the coverings are contemplated for use to make the covering of the invention.

In some embodiments, biodegradable polymers have diphenol monomer units that are copolymerized with an appropriate chemical moiety to form a polyarylate, a polycarbonate, a polyiminocarbonate, a polyphosphonate or other class of polymer.

For example, suitable biodegradable tyrosine-derived polyarylates include those described in U.S. Pat. Nos. 5,099,060; 5,216,115; 5,317,077; 5,587,507; 5,658,995; 5,670,602; 6,048,521; 6,120,491; 6,319,492; 6,475,477; 6,602,497; 6,852,308; 7,056,493; RE37,160E; and RE37,795E; as well as those described in U.S. Patent Application Publication Nos. 2002/0151668; 2003/0138488; 2003/0216307; 2004/0254334; 2005/0165203; and those described in PCT Publication Nos. W099/52962; WO 01/49249; WO 01/49311; WO03/091337. These patents and publications also disclose other useful polymers containing tyrosine-derived diphenol monomer units or other diphenol monomer units, including polyarylates, polycarbonates, polyiminocarbonates, polythiocarbonates, polyphosphonates and polyethers.

Likewise, the foregoing patents and publications describe methods for making these polymers, some methods of which may be applicable to synthesizing other biodegradable polymers. The foregoing patents and publications also describe blends and copolymers with polyalkylene oxides, including PEG. All such polymers are contemplated for use in the present invention.

The representative structures for the foregoing polymers are provided in the above-cited patents and publications which are incorporated herein by reference in their entireties. Polyarylates are among the preferred materials because their physical characteristics can match those of silicone. Additionally, polyarylates are preferred because their range of Tg can be manipulated to form a film for coating an outer-shell of a breast implant.

Abbreviations used herein for naming polymers and the subunits thereof include B, 4-hydroxybenzoic acid; Bn or Bz, benzyl; D or DAT, desaminotyrosine or desaminotyrosyl; DATE, desaminotyrosine ethyl ester; E or Et, ethyl; glu, glutarate; M or Me, methyl; PEG, polyethylene glycol; Succ, succinate; and T, tyrosine.

As used herein, polymers based on diphenol monomer units have two-part names. The first part identifies the diphenol moiety and the second part identifies the group with which the diphenol moiety is copolymerized. The names are written in the form poly(diphenol diacid), poly(diphenol carbonate), poly(diphenol iminocarbonate), etc.

The diphenol moiety is generally named for its three components, the two aromatic ring moieties and the tyrosine ester moiety. For example, DTE is desaminotyrosyl-tyrosine ethyl ester; DTBn is desaminotyrosyl-tyrosine benzyl ester. When a free acid is present (rather than an ester), the name for a third component is omitted. Thus, DT is the corresponding free acid form, namely desaminotyrosyl-tyrosine. BTE is the diphenol monomer 4-hydroxy benzoic acid-tyrosine ethyl ester; BT is the corresponding free acid form, namely 4-hydroxy benzoic acid-tyrosine.

The second part of the name identifies the group with which the diphenol moiety is polymerized, such as the diacid, the carbonate, the iminocarbonate and the like. Hence, specific examples include poly(DTE glutarate), poly(DTBn carbonate) and the like.

If a mixture of diphenol moieties or of copolymerized groups (such as two diacids) are present in the polymer, then that part of name may includes the designation “co” or may have a hyphen, along with an indication of percentage of one of the two moieties. For example, poly(DTE:10DT-co-succinate) and poly(DTE-10-DT succinate) are used interchangeably to mean a polymer made by copolymerizing a mixture of 90% desaminotyrosyl-tyrosine ethyl ester and 10% desaminotyrosyl-tyrosine with the diacid succinic acid. An example of a mixed diacid is poly(DTE-co-50:50 PEG-bis-succinate adipate).

Additional preferred polyarylates are random copolymer of desaminotyrosyl-tyrosine (DT) and an desaminotyrosyl-tyrosine ester (DT ester), wherein the copolymer comprises from about 0.001% DT to about 80% DT and the ester moiety can be a branched or unbranched alkyl, alkylaryl, or alkylene ether group having up to 18 carbon atoms, any group of which can optionally have a polyalkylene oxide therein. Similarly, another group of polyarylates are similar to the foregoing but the desaminotyrosyl moiety is replaced by a 4-hydroxybenzoyl moiety. Preferred DT or BT contents include those copolymers with from about 1% to about 30%, from about 5% to about 30% from about 10 to about 30% DT or BT. Preferred diacids (used in forming the polyarylates) include succinic, glutaric, sebacic, adipic and glycolic acid as well as PEG or other PAOs and polyethylene glycol diacids such as the polyethylene glycol-bis-alkyl diacids described in U.S. Pat. No. 7,271,234.

Useful tyrosine-derived polyarylates are the DTE-DT succinate family of polymers, e.g., those polymers having from 0-50%, 5-50%, 5-40%, 1-30% or 10-30% DT, including but not limited to, about 1, 2, 5, 10, 15, 20, 25, 27.5, 30, 35, 40%, 45% and 50% DT.

Some polyarylates have inherent microtexturing, which may be desirable in forming a temporarily-textured outer surface on the breast implant.

Any drug, biological agent, or active ingredient that is compatible with the process of preparing the covering of the invention can be incorporated into one or more of the polymeric biodegradable layers.

Doses of such drugs and agents are known in the art. Those of skill in the art can readily determine the amount of drug or agent desired for delivery, and calculate the amount of that should be loaded into the polymeric layers of the covering for a breast implant of a particular volume.

The drug or drugs are retained in the covering or coating and in essence provide for a topical application of the drug or drugs. The active agents may be a mixture of leukotriene antagonist, antibiotic and calcium channel blocker that is released to the surface of the breast implant and the body cavity in which the implant is implanted. The drug or drugs mixture can be applied in a time-release fashion by adhering this mixture to a lattice or in a layered fashion that allows for controlled release from one week to 18 months. Further, this combination of medications in the lattice or clathrate compound or biodegradable polymeric material can be applied to a smooth walled implant, textured wall implant or dissolvable textured implant. Further, the clathrate or biodegradable polymer can be designed to have three dimensional properties to create a textured surface, eliminating the necessity for another substance to create the three dimensional coating. As a result the clathrate or biodegradable polymer will release the mixture of drugs and then dissolve away.

Drug elution times can be determined based on the drug and its time course of action, which generally and more preferably are over the course of 3 to 100 days. For example, antibiotic activity for 7-10 days (or more) can be sufficient to prevent or reduce colonization of implants, thereby preventing or reducing capsular contracture (or its overall incidence as could be assessed, for example, in a clinical trial).

In accordance with the invention, the drugs and biologically-active agents for formulation into the polymeric layers of the coverings include, but are not limited to, antibiotics, anti-inflammatory agents, fibrosis-inhibiting agents, anti-scarring agents, leukotriene inhibitors or antagonists, cell growth inhibitors, calcium channel blockers, or other agents effective to treat various conditions associated with implant surgery and recovery.

As used herein, the term “drug” or “drugs” is used to include all types of therapeutic agents, whether small molecules or large molecules such as proteins, nucleic acids and the like. The drugs of the invention can be used alone or in combination.

Certain preferred agents include antibiotics; leukotriene antagonists, such as zafirlukast (Accolate®), montelukast (Singulair®), pranlukast and zileuton; antineoplastic agents, such as 5-fluoruricil; nitric oxide producing agents, such as L-arginine; calcium-channel blockers, such as verapamil; TNF; interleukins; interferons; paclitaxel or other chemotherapy agents; 2-mercaptoethanesulfonate; antifungal agents; as well as any other agent, especially those that are known to for their ability to reduce capsular contracture.

Examples of non-steroidal anti-inflammatory agents include, but are not limited to, acetaminophen, aspirin, celecoxib, diclofenac, diflunisal, flurbiprofen, ibuprofen, indomethacin, ketoprofen, ketorolac, meclofenamate, meloxicam, methyl salicylate, nabumetone, naproxen, oxaprozin, piroxicam, sulindac, tolmetin and trolamine.

Examples of antimicrobial drugs include, but are not limited to:

aminoglycosides, such as amikacin, gentamicin, kanamycin, neomycin, streptomycin, and tobramycin;

antibiotics, such as bacitracin, clindamycin, daptomycin, lincomycin, linezolid, metronid, polymyxin, rifaximin, vancomycin;

cephalosporins, such as cephazolin or cephalexin;

macrolide antibiotics, such as erythromycin, azithromycin and the like;

β-lactam antibiotics, such as penicillins;

quinolones, such as ciprofloxacin;

sulfonamides, such as sulfadiazine;

tetracyclines, such as minocycline and tetracycline; and

other antibiotics, such as rifampin, triclosan and chlorhexidine.

Other drugs, besides those discussed above, that can be incorporated into the polymeric layers of the coverings of the invention, include, but are not limited to, acyclovir, cephradine, malphalen, procaine, ephedrine, adriamycin, daunomycin, plumbagin, atropine, quinine, digoxin, quinidine, biologically active peptides, cephradine, cephalothin, cis-hydroxy-L-proline, melphalan, penicillin V, nicotinic acid, chemodeoxycholic acid, chlorambucil and anti-neoplastic agents such as paclitaxel, sirolimus, 5-flurouracil and the like. Other drugs include those that act as angiogenensis inhibitors or inhibit various growth factors such as epidermal growth factor, PDGF, VEGF, FGF (fibroblast growth factor) and the like. These drugs include anti-growth factor antibodies (neutrophilin-1) and growth factor receptor-specific inhibitors such as endostatin.

As also mentioned above, any other drug or active therapeutic agent may be used, depending on the condition of the subject into which the implant is implanted, and particularly those that reduce, prevent or treat capsular contraction or other capsule formation can be incorporated into the covering of the invention.

Preferred antimicrobial agents of the invention include bacitracin, cephalexin and gentamicin, alone or in combination.

Examples of leukotriene inhibitors or leukotriene receptor antagonists include, but are not limited to, leukotriene receptor antagonists such as acitazanolast, iralukast, montelukast, pranlukast, verlukast, zafirlukast, and zileuton.

Once the drug or combination of drugs, and their appropriate concentration and dosage have been selected, the drug or drugs are mixed with the material used to form the one or more layers of the covering, which is provided in liquid form, typically in a solution with a biocompatible solvent appropriate for the particular clathrate compound or biodegradable polymer or polymer precursor. The drug-covering polymer or polymer percursor composition is then applied either to an appropriately-shaped mandrel or mold to form a cover, or applied to the implant directly to form an implant coating, by any of the above-mentioned methods. The resulting product is then dried, typically with mild heating, to drive off the solvent. This produces the cover for separately encasing the implant or the coated implant with the drug elutably contained within the covering. If multiple layers are desired, which may have the same or different concentrations of the same drug or drug combination or different drugs or drug combinations, the process is repeated for each layer. The inner or outer surface or both, of the covering may be textured, as can the outer surface of the coating, as desired, using a textured mandrel or mold, air or other gas spraying or the like.

After manufacture, the coverings of the invention in the form of covers can be sterilized and packaged and for assembly onto a breast implant immediately prior to surgery. Alternatively, the coverings of the invention can be assembled onto the breast implant, either in the form of covers or coatings, sterilized and packaged at the time of manufacture, so that a completed breast implant assembly is delivered to the surgical suite. In any event, sterile gloves and sterile and atraumatic instruments should be used when handling the coverings to provide a sterile breast implant assembly of the implant and the covering.

Once form-fitted with a cover or as a coated implant, the implant assembly can be inserted into the subject, typically a human, and usually a woman, but also including a man, but the subject could also be any desired animal, using standard breast reconstructive or augmentative surgical techniques. Breast reconstruction usually occurs following complete mastectomy but can be also be done for a partial mastectomy usually in women, but also in men, as well as to correct or repair congenital deformities or traumatic injury to the breast. Breast augmentation is typically done by women for cosmetic reasons or by men regarding transgender surgery.

Another aspect of the invention is directed to a kit comprising a breast implant and any of the biodegradable covers of the invention. The cover in the kit is size matched to the implant supplied with the kit. The kits are sterile. The kits optionally contain instructions for inserting the accompanying implant into the cover or for handling and surgically implanting the breast implant assembly in the subject. At the time of surgery, the kits are opened and the implant is inserted in the covering as described herein.

Alternatively, the kit can consist of a breast implant assembly of the invention that comprise the biodegradable covering of the invention containing or wrapped around a breast implant or a breast implant coated with the drug-containing clathrate compound or biodegradable polymeric material. As with the previously-described kits, the coverings and implants of the assembly are appropriately sized matched and sterile.

A further aspect of the invention is directed to a method for reducing post-surgical complications from breast augmentation or breast reconstruction in a subject which comprises surgically implanting a breast implant assembly of the invention into the subject. The breast implant assembly is used in standard surgical breast augmentation or reconstruction procedures and do not lead to any major changes or complications in those procedures.

All references, patents, patent applications or other documents cited are hereby incorporated by reference herein in their entirety.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A biodegradable covering for a breast implant, the covering comprising at least one biodegradable polymer layer dimensioned and shaped to cover the breast implant, the covering containing at least one drug to at least minimize at least one of infection and capsular contraction.

2. The covering of claim 1 which comprises a plurality of biodegradable layers.

3. The covering of claim 1, wherein at least one layer comprises more than one drug.

4. The covering of claim 1, wherein the at least one drug is selected from the group consisting of an antimicrobial agent, anti-inflammatory agent, anti-scarring agent, anti-neoplastic agent, a calcium channel blocker and leukotriene inhibitor.

5. The covering of claim 4, wherein the at least one drug is an antimicrobial agent.

6. The covering of claim 5, wherein the antimicrobial agent is selected from the group consisting of bacitracin, cephalexin and gentamicin, and mixtures thereof.

7. The covering of claim 5, wherein the at least one drug is a leukotriene inhibitor.

8. The covering of claim 7, wherein the leukotriene inhibitor is a leukotriene receptor antagonist selected from the group consisting of acitazanolast, iralukast, montelukast, pranlukast, verlukast, zafirlukast, and zileuton.

9. The covering of claim 1, wherein the covering has a smooth outer surface.

10. The covering of claim 1, wherein the covering has a textured outer surface.

11. A kit comprising a breast implant and the biodegradable covering of claim 1.

12. A breast implant assembly comprising a biodegradable covering of claim 1 containing a breast implant.

13. A method for reducing at least one post-surgical indication from breast augmentation or breast reconstruction in a subject, the method comprising surgically implanting into the subject a breast implant assembly of claim 12.

14. The method of claim 13, wherein the indication is selected from the group consisting of infection, inflammation, capsular contracture and scarring.
